(19) United States
(12) Patent Application Publication
(10) Pub. No.: US 2025/0186361 A1
(43) Pub. Date: Jun. 12, 2025

(54) METHOD AND DEVICE FOR THE PRODUCTION OF A CARRIER

(71) Applicant: QUANTOOM BIOSCIENCES S.A., Nivelles (BE)

(72) Inventors: Aurélien VANDER STRAETEN, Watermael-Boitsfort (BE); José CASTILLO, Nivelles (BE)

(21) Appl. No.: 18/984,458

(22) Filed: Dec. 17, 2024

Related U.S. Application Data
(63) Continuation of application No. PCT/EP2023/066091, filed on Jun. 15, 2023.

(30) Foreign Application Priority Data
Jun. 17, 2022 (EP) 22179735.0
Nov. 17, 2022 (EP) 22208001.2

(51) Int. Cl. A61K 9/51; B01D 61/36; B01F 23/40; B01F 23/80; B01F 33/30

(52) U.S. Cl. CPC A61K 9/5192; A61K 9/5123; B01D 61/362; B01D 61/366; B01F 23/49; B01F 23/806; B01F 23/808; B01F 33/30

(57) ABSTRACT

The current invention relates to a method for manufacturing one or more carriers with one or more active pharmaceutical ingredients, wherein said method comprises mixing at least a solution comprising one or more active pharmaceutical ingredients with one or more organic solvents comprising one or more lipids and/or polymers, thereby forming a formulation comprising one or more carriers, and subsequently removing at least part of said one or more organic solvents by a pervaporation step. In a second aspect, said invention also relates to a system for manufacturing such carriers, wherein said system comprises a pervaporation device for removal of one or more organic solvents from a formulation comprising one or more carriers.

### CROSS-REFERENCE

This application is a Continuation of International Application No. PCT/EP2023/066091, filed Jun. 15, 2023, which claims the benefit of EP Application Serial No. 22179735.0, filed Jun. 17, 2022, and EP Application Serial No. 22208001.2, filed Nov. 17, 2022, each of which is incorporated herein by reference in its entirety.

### FIELD OF THE INVENTION

This invention relates to a method and a system for the production of a carrier as a therapeutic delivery system.

### BACKGROUND

Carriers, such as lipid-based carriers (e.g. lipid nanoparticles (LNPs)) or polymeric nanocarriers, are the most widely used gene delivery systems. For their fabrication, an aqueous phase containing the genetic material is mixed with an organic phase containing a lipid, a polymer or a mix of lipids and/or polymers. This process results in the encapsulation of the genetic material inside a nanoparticle or nanocarrier. After their fabrication the organic solvent must be removed to meet the regulatory requirements for medicinal products. Solvent extraction and exchange is frequently performed by Tangential Flow Filtration (TFF) with several consecutive cycles of concentration and dilution with an aqueous buffer until the desired residual solvent concentration is in compliance with current regulatory requirements.

However, the TFF operation can result in considerable shear stress with an adverse impact on the quality of the nanocarriers.

Furthermore, TFF is a limiting step in the nanocarrier production process as it requires dilution of the solution and increases the volume of the solution to be processed which leads to increased waste. Such a TFF step must be operated in batch (as opposed to a continuous processing model), with several disadvantages, such as an increased equipment footprint, a longer total processing time and increased human intervention. Finally, traditional TFF operations are difficult to scale up.

Therefore, there is a need for a more efficient method for separating organic solvent from produced nanocarriers, preferably one which can be operated in continuous mode.

Pervaporation can be used to selectively remove substances from a solvent mixture (including azeotropic mixtures). For example, hydrophilic membranes can be used to remove water from organic solvents and can provide the organic solvent in a higher purity than conventional means. One particular use is to purify compounds which form azeotropic mixtures with water, such as ethanol and isopropanol.

US 2008/171078 A1 presents liposomes as a delivery method for transporting a payload to a patient. It also reveals a specific technique for producing liposomes of desired sizes, by utilizing evaporation of a solvent from a two-phase liquid emulsion. In particular, this method involves creating droplets of a first liquid, which contains one or more lipids, by injecting it into a continuous flow of an aqueous second liquid. It is crucial for the success of the method that the first liquid remains sparingly soluble in the second liquid, without dissolving to form droplets. These droplets must be maintained within the second liquid during the process. Downstream, the first liquid is subsequently evaporated from these droplets (for example, using a membrane pervaporation unit). This evaporation step leads to the removal of the first liquid allowing the lipids to come into contact with the aqueous second liquid. At this point, the lipids spontaneously self-assemble and arrange themselves to form liposomes. The diameters of these liposomes are closely related to the initial volume of the droplets and the concentration of lipids in the first liquid droplets.

U.S. Pat. No. 5,110,475 A relates to a process that is suitable in the manufacture of phase mixtures which contain liposomes or micelles. These mixtures, as mentioned above, require dissolving the compounds forming liposomes and/or micelles in an organic phase and making an emulsion (droplets in aqueous phase) and removal of solvent from such droplets.

Hydrophilic membranes can be used for the removal of water from fermentation broths or from condensation reactions such as esterification. Hydrophobic membranes can also be used for the removal of organic solvent. Polymeric membranes, such as polydimethylsiloxanes (PDMS) membranes, are as well widely used. They are generally less-selective but good alternatives for the removal of organic solvent from mixtures with aqueous solvent.

There is a need and potential to improve the current methods to remove solvent from the medium that produced nanocarriers are in.

### SUMMARY OF THE INVENTION

The present invention and embodiments thereof serve to provide a solution to one or more of above-mentioned disadvantages. To this end, the present invention relates to a method for manufacturing a formulation of one or more lipid nanoparticles, polymeric, or hybrid carriers comprising one or more active pharmaceutical ingredients, said method comprises mixing a solution of one or more active pharmaceutical ingredients with one or more organic solvents comprising one or more lipids and/or one or more polymers, and subsequently removing at least part of said one or more organic solvents by a pervaporation step, according to claim 1.

Preferred embodiments of the method are shown in any of the claims 2 to 18.

In a second aspect, the present invention relates to a system for manufacturing a formulation comprising one or more of carriers with one or more active pharmaceutical ingredients, according to claim 19 and dependent claims 20 to 24.

Particularly, the present invention relates to a system for manufacturing a formulation comprising one or more carriers with one or more active pharmaceutical ingredients, wherein, said system comprises one or more mixing units suited to mix at least one solution comprising one or more active pharmaceutical ingredients with a composition of one or more organic solvents comprising one or more lipids and/or one or more polymers thereby forming a formulation comprising one or more carriers, wherein said one or more mixing units are fluidly connected to a pervaporation device for removal of at least a part of the organic solvents from said formulation.

Summarized, the invention pertains at least to the following embodiments:

1. A method for manufacturing a formulation of one or more carriers comprising one or more active pharmaceutical ingredients, said method comprises mixing at least:
a. a solution of one or more active pharmaceutical ingredients; with
b. one or more organic solvents comprising one or more lipids and/or one or more polymers,
and subsequently removing at least part of said one or more organic solvents by a pervaporation step.
   2. The method according to embodiment 1, wherein said carriers are lipid-based carriers, polymeric carriers or hybrid carriers.
   3. The method according to any of the previous embodiments, wherein said active pharmaceutical ingredient is chosen from a polynucleotide, a chemical compound, a polypeptide or any combination thereof.
   4. The method according to any of the previous embodiments, wherein said active pharmaceutical ingredient is a polynucleotide.
   5. The method according to any of the previous embodiments, wherein a further, preferably aqueous, solution comprising one or more polymers is mixed with the solution of one or more active pharmaceutical ingredients and the mixture of one or more organic solvents and one or more lipids and/or polymers.
   6. The method according to any of the previous embodiments, wherein said pervaporation process is a membrane separation process, preferably by making use of one or more gas-permeable membranes.
   7. The method according to embodiment 6, wherein said membrane is a porous membrane.
   8. The method according to any of the previous embodiments, wherein said pervaporation step occurs in a pervaporation device comprising at least one separation membrane, and wherein a vacuum is applied and/or a purge gas is introduced, thereby allowing the one or more organic solvents to diffuse through said membrane.
   9. The method according to any of the previous embodiments, wherein during said pervaporation process the flow rate is between 10 μL/min and 10 L/min, preferably between 10 μL/min and 500 mL/min.
   10. The method according to any of the previous embodiments, wherein said one or more organic solvents are chosen from the group of alcohols, preferably from the group of ethanol, methanol, propanol, isopropanol, butanol or mixtures thereof.
   11. The method according to any of the previous embodiments, wherein said mixing occurs by means of microfluidic mixing.
   12. The method according to any of the previous embodiments, wherein said carriers have a size of at most 10 microns, more preferably at most 1 micron.
   13. A system for manufacturing a formulation comprising one or more carriers with one or more active pharmaceutical ingredients, wherein, said system comprises one or more mixing units suited to mix at least one solution comprising one or more active pharmaceutical ingredients with a composition of one or more organic solvents comprising one or more lipids and/or one or more polymers thereby forming a formulation comprising one or more carriers, wherein said one or more mixing units are fluidly connected to a pervaporation device for removal of at least a part of the organic solvents from said formulation.
   14. The system according to embodiment 13 wherein said mixing unit is a microfluidic mixing unit.
   15. The system according to embodiment 14 wherein said microfluidic mixing unit comprises an assembly of a plurality of microfluidic mixers in parallel, preferably 5 to 20 of said mixers.
   16. The system according of any of the embodiments 13 to 15 wherein said pervaporation device comprises at least one separation membrane, preferably sandwiched between metal or plastic plates.
   17. The system of any of the embodiments 13 to 16 wherein said pervaporation device comprises a plurality of stacks of pervaporation units wherein said each unit comprises a separation membrane sandwiched between one or more metal plates.
   18. The system of any of the embodiments 13 to 17 further comprises one or more condensers.

### DESCRIPTION OF FIGURES

FIG. 1A shows the size and PDI of LNPs that were pervaporated at different total flow rates (TFR) and different temperatures FIG. 1B Encapsulation efficiency of LNPs that were pervaporated at different total flow rates (TFR) and different temperatures.

FIG. 2A shows measured vs. predicted residual EtOH content for LNP system pervaporation. FIG. 2B shows measured vs. predicted pervaporation fraction for binary system pervaporation.

FIG. 3 shows luciferase activity of HEK293 cell transfected with LNPs encapsulating RNA encoding for Firefly Luciferase obtained by pervaporation at different total flow rates (TFR) and different temperatures. Ctl shows LNPs not pervaporated and dialyzed using a standard laboratory procedure.

FIG. 4 and FIG. 5 show a possible embodiment of a system according to the current disclosure.

FIG. 6A shows a schematic overview of laminar microfluidic mixing unit, whereas FIG. 6B shows a schematic overview of a chaotic microfluidic mixing unit.

FIG. 7A shows a schematic overview of a pervaporation unit combined with a condenser, whereas FIG. 7B shows a setup without a condenser.

FIG. 8A-FIG. 8E shows different schematic set-ups of possible embodiments of the system of the current invention.

### DETAILED DESCRIPTION OF THE INVENTION

The present invention discloses a method for manufacturing a formulation comprising one or more carriers comprising, enclosing, bound to, or adsorbing one or more active pharmaceutical ingredients (API). An example of such carrier is a lipid nanoparticle (LNP). The method comprises mixing two or more liquids for the formation of carriers. For example by mixing a solution comprising one or more APIs with one or more solvents comprising one or more lipids and/or polymers, thereby forming a formulation comprising one or more carriers, and subsequently removing at least part of said one or more organic solvents by a pervaporation step. The present disclosure also aims to resolve at least some of the problems and disadvantages discussed below.

After fabrication of the carriers such as nanocarriers by mixing an aqueous solution and organic solvent, the organic solvent must be removed. The traditional methods including TFF for removal of organic solvent from a solution comprising carriers can result in considerable shear stress with an adverse impact on the quality of the carriers and requires use of large buffer volumes to achieve the targeted dilution. Traditional methods mostly need to be operated in batches which brings several other disadvantages, such as an increased equipment footprint, a longer total processing time, increased human intervention and of course difficulty to scale up the production.

### Definitions

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

“Carrier”, “nanocarrier” is material being used as a transport module for another substance, such as a drug. The term “carrier” is used if the diameter of the material is ranging from 1 nm to 10 um whereas the term “nanocarrier” is used to define materials with ranging sizes of diameter 1 nm to 1000 nm. Therefore, the term “carrier” as used herein includes the “nanocarriers”. The term “carrier” as used herein also refers to nanocarriers, nanoparticles, nanoparticle drug carriers. Commonly used carriers include micelles, polymers, carbon-based materials, liposomes and other substances. Other non-limiting examples of carriers include polymer-based carriers, polymer conjugates, polymeric nanoparticles, lipid-based carriers, polymer lipid hybrid carriers, dendrimers, carbon nanotubes, gold nanoparticles and the silica nanoparticle.

Non limiting examples of lipid-based carriers includes liposomes, solid-lipid Nanoparticles, non-structures lipid carriers, nanoemulsions.

Non limiting examples of polymer-based carriers includes polymer nanoparticles, polymer micelles, polymer vesicles, dendrimers, metal-organic frameworks.

Non limiting examples of polymer lipid hybrid carriers includes polymer core-lipid shell nanoparticles, hollow core/shell lipid-polymer-lipid hybrid nanoparticles, Lipid bilayer-coated polymeric particle, Mixed lipid polymer nanoparticles.

As used herein, the term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

As used herein, the phrase “lipid nanoparticle (LNP)” refers to a nanosized vesicle or carrier comprising one or more lipids (e.g., cationic and/or non-cationic lipids).

As used herein, the term “cationic” means that the respective structure bears a positive charge, either permanently or not permanently but in response to certain conditions such as pH. Thus, the term “cationic” covers both “permanently cationic” and “cationisable”.

As used herein, the phrase “cationic lipid” refers to any of a number of lipid species that carry a net positive charge at a selected pH, such as physiological pH.

The term “cationisable” as used herein means that a compound, or group or atom, is positively charged at a lower pH and uncharged at a higher pH of its environment. Also, in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. For example, in some embodiments, if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions of the cell in vivo. In other embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7.

The term “permanently cationic” as used herein will be recognized and understood by the person of ordinary skill in the art, and means, for example, that the respective compound, or group or atom, is positively charged at any pH value or hydrogen ion activity of its environment. Typically, the positive charge results from the presence of a quaternary nitrogen atom. Where a compound carries a plurality of such positive charges, it may be referred to as permanently polycationic, which is a subcategory of permanently cationic.

In the context of the present disclosure, the term “PEGylated lipid” is meant to be any suitable lipid modified with a PEG (polyethylene glycol) group.

In the context of the present disclosure, the term “sterol”, also known as steroid alcohol, is a subgroup of steroids that occur naturally in plants, animal and fungi, or can be produced by some bacteria. In the context of the present disclosure, any suitable sterol may be used, such as selected from the list comprising cholesterol, ergosterol, campesterol, oxysterol, antrosterol, desmosterol, nicasterol, sitosterol and stigmasterol; preferably cholesterol.

As used herein, the term “aqueous” refers to a composition comprising in whole, or in part, water.

As used herein, the term “lipid solution” refers to a composition comprising in whole, or in part, an organic solvent having a lipid.

As used herein, the terms “polynucleotide” and “nucleic acid” are used interchangeably to refer to genetic material (e.g., DNA or RNA), and when such terms are used with respect to the lipid nanoparticles, they generally refer to the genetic material encapsulated by such lipid nanoparticles.

As used herein, the terms “API”, “active pharmaceutical ingredient”, and “active pharmaceutical agent” are used interchangeably to refer to a biologically active compound. API generally refers to the substances in pharmaceuticals that are responsible for the beneficial health effects experienced by consumers. Examples of APIs include, without limitation a nucleic acid, a polynucleotide, such as RNA and DNA, a peptide, a polypeptide, an excipient, a chemical substance and intermediates, an antibody, an antibody fragment, an antibody-like protein scaffold, a protein, a peptidomimetic, an aptamer, a photoaptamer, a spiegelmer or any combination thereof.

The terms “DNA” or “DNA molecule” are used herein to generally refer to any type DNA. Non-limiting example of DNA includes any (single-stranded or double-stranded) DNA, preferably, without being limited thereto, e.g. genomic DNA, single-stranded DNA molecules, double-stranded DNA molecules, coding DNA, DNA primers, DNA probes, immunostimulatory DNA, a (short) DNA oligonucleotide ((short) oligodesoxyribonucleotides), viral DNA, or a combination thereof.

The terms “RNA” or “RNA molecule” are used herein to generally refer to any type RNA. Non-limiting example of RNA includes long-chain RNA, coding RNA, non-coding RNA, long non-coding RNA, single stranded RNA (ssRNA), double stranded RNA (dsRNA), linear RNA (linRNA), circular RNA (circRNA), messenger RNA (mRNA), self-amplifying mRNA (SAM), Trans amplifying mRNA, RNA oligonucleotides, antisense oligonucleotides, small interfering RNA (SIRNA), small hairpin RNA (shRNA), antisense RNA (asRNA), CRISPR/Cas9 guide RNAS, riboswitches, immunostimulating RNA (isRNA), ribozymes, aptamers, ribosomal RNA (rRNA), transfer RNA (tRNA), viral RNA (vRNA), retroviral RNA or replicon RNA, small nuclear RNA (snRNA), small nucleolar RNA (snoRNA), microRNA (miRNA), transcription start site-associated (TSSa-)RNAs, upstream antisense (ua) RNAs, promoter upstream transcripts (PROMPTs), or a combination thereof.

According to the present disclosure, the term “RNA” includes and preferably relates to “mRNA” which means “messenger RNA” and relates to a “transcript” which may be produced using DNA as template and encodes a peptide, a polypeptide, or protein. mRNA typically comprises a 5′ untranslated region (5′-UTR), a protein or peptide coding region and a 3′ untranslated region (3′-UTR). mRNA has a limited halftime in cells and in vitro. Preferably, mRNA is produced by in vitro transcription using a DNA template. In one embodiment of the disclosure, the RNA is obtained by in vitro transcription or chemical synthesis. The in vitro transcription methodology is known to the skilled person. For example, there is a variety of in vitro transcription kits commercially available. Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

An active ingredient or principle is any component that provides biologically active or other direct effect in the diagnosis, cure, mitigation, treatment, or prevention of disease or to affect the structure or any function of the body of humans or animals.

### Description

In a first aspect, a method for manufacturing a formulation of one or more lipid nanoparticles, polymeric, or hybrid carriers with one or more active pharmaceutical ingredients (API) is disclosed. In embodiments said method comprises mixing a solution of one or more APIs and a mixture of one or more organic solvents and one or more lipids and/or polymers thereby forming a formulation comprising one or more carriers, and subsequently removing at least part of said one or more organic solvents by a pervaporation step.

In embodiments, said method comprises mixing two or more liquids. In embodiments, the method comprises mixing an aqueous solution comprising one or more polynucleotides of interest with one or more lipids and/or polymers and one or more organic solvents, thereby forming a formulation comprising one or more carriers, and subsequently removing at least part of said one or more organic solvents by a pervaporation step.

In embodiments, said method further comprises mixing a further, preferably aqueous, solution comprising one or more polymers with the solution of one or more active ingredients and the mixture of one or more organic solvents and one or more lipids and/or polymers. In an embodiment, to prepare carriers comprising pharmaceutical ingredients, the lipid components are first dissolved in an organic solvent such as ethanol and then mixed with an aqueous solution of pharmaceutical ingredients to allow particle formation by nanoprecipitation. In embodiments, aqueous solution of pharmaceutical ingredients is an acidified aqueous solution.

In an embodiment, the formulation of carriers involves the rapid microfluidic mixing of an organic phase comprising one or more lipids with an aqueous phase containing the active pharmaceutical ingredients, such as a polynucleotide, thereby forming particles, such as nanoparticles. These particles organize into a dense structure, wherein the core contains the active pharmaceutical ingredients electrostatically complexed with one or more lipids.

In an embodiment, laminar microfluidic mixing is used for mixing. In another embodiment, chaotic microfluidic mixing is used for mixing.

In the embodiments, the mixing of an organic phase comprising one or more lipids with an aqueous phase, preferably containing active pharmaceutical ingredients, results in a single-phase liquid mixture comprising the formed carriers.

In conventional methods of producing lipid-based carriers like liposomes, it is crucial to remove the organic solvent from an emulsion in the aqueous phase to ensure proper formation of lipid vesicles.

The organic solvent is used to dissolve the lipids and create a homogeneous lipid film. However, lipids are hydrophobic molecules and tend to aggregate in organic solvents rather than dispersing evenly in water, leading to a multi-phase liquid mixture resembling an emulsion with droplets of lipids in the aqueous phase. To form liposomes, it is essential to remove the organic solvent from the droplets in the emulsion and replace it with an aqueous phase. This can be achieved through evaporation, for example, using a membrane pervaporation unit. The evaporation step allows the lipids to concentrate in the first liquid, forming a thin film and coming into contact with the second liquid. As a result, the lipids spontaneously self-assemble to form liposomes.

In the present disclosure, the method focuses on the formation of lipid nanoparticles (LNPs). LNPs have a different structure than liposomes and their production does not rely on forming droplets of the first liquid in a mixture of two non-miscible liquids. Instead, LNPs are suspended in a single-phase mixture where the individual molecules of the organic solvent and aqueous solution disperse evenly. For instance, in specific embodiments, ethanol and water are used as the organic solvent and aqueous solution, respectively, with ethanol dispersed in the mixture through vigorous mixing or sonication. LNPs which are not soluble in such single-phase mixture comprising organic and aqueous solvents will be formed. The common practice to remove the solvent in this step of LNP production is Tangential Flow Filtration (TFF), involving several cycles of concentration and dilution with an aqueous buffer until the residual solvent concentration meets regulatory requirements. However, TFF can impose shear stress on the nanocarriers, negatively impacting their quality. The inventors provide a novel solution by proposing the use of pervaporation to remove the solvent from the single-phase solution to obtain purified LNPs. This alternative method avoids the adverse effects of shear stress associated with TFF, ensuring the quality and integrity of the nanocarriers.

As such, in an embodiment, the method further comprises yielding a single-phase liquid mixture comprising said lipid nanoparticles, polymeric, or hybrid carriers resulted from (a). In embodiments, organic solvent and aqueous solution forms a single-phase mixture liquid.

In embodiments, the individual molecules of the organic solvent and aqueous solution disperse evenly or at least partially evenly.

In embodiments, the organic solvent and water are miscible, which means they can mix together at least in specific portions, preferably in all portions. In embodiments, the organic solvent and water are miscible following the mixing step according to the disclosed method.

In an embodiment, the encapsulation efficiency of one or more active pharmaceutical ingredients is at least 60%, preferably at least 65%, preferably at least 70%, preferably at least 75%, more preferably at least 80%, more preferably at least 85%, more preferably at least 90%, more preferably at least 95%.

In embodiments, said method comprises introducing the formulation comprising one or more carriers into a pervaporation device comprising at least one separation membrane, and applying a vacuum and/or introducing a purge gas, thereby allowing the one or more organic solvents to diffuse through said membrane. In some embodiments, only a vacuum is applied, in other embodiments, only a purge gas is used and in other embodiments, both a vacuum and purge gas is used.

In an embodiment, said removing at least part of said one or more organic solvents occurs by means of a pervaporation process. Pervaporation is a membrane separation process that is an energy-efficient combination of permeation and evaporation. It is applied to the removal of volatile compounds out of solutions through a selective membrane. By creating a vacuum or introducing a flow of purge gas on one side of a membrane, volatile compounds in a liquid flow on the other side will diffuse through this membrane. In some embodiments, to a smaller extent, the aqueous phase may also diffuse through said membrane. Compared to traditional Tangential Flow Filtration (TFF), the pervaporation step does not require a buffer and therefore reduces process footprint and waste. As such, in an embodiment of the method, said pervaporation process does not comprise utilizing a buffer. It is also lower in shear stress.

In an embodiment, said method further comprises reducing shear stress on said one or more carriers. In an embodiment, said method further comprises reducing shear stress by at least 10%, preferably by at least 20%, more preferably by at least 30%, more preferably by at least 40%, on said one or more carriers. As described above, “carriers” as used herein can comprise LNPs, polymeric, or hybrid carriers.

The pervaporation can be operated in a continuous fashion, provided that the surface of the membrane and the surface to volume ratio are large enough. Continuous processing confers several advantages, such as a decrease in equipment footprint, automatability and an ability to increase scale by scale-out of compact manufacturing lines, or extension of the processing time.

In an embodiment, said one or more organic solvents are chosen from the group of alcohols. Non-limiting examples of said alcohols can be ethanol, methanol, propanol, isopropanol, butanol, or mixtures thereof.

In an embodiment, said pervaporation process is a membrane separation process, preferably by making use of one or more gas-permeable membranes.

In an embodiment, said membrane is a porous membrane. In specific embodiments said membrane is a silicone such as a polydimethylsiloxane (PDMS) membrane.

In embodiments, the membranes are made of hybrid materials comprising PDMS. In embodiments, membranes further comprise one or more supporting materials which can be made of cellulose, PET (polyester) or alike. In further embodiments, membranes comprise an intermediate layer made of a type of phosphatidylinositol (PI).

In embodiments, said method comprises a pervaporation step. The pervaporation step occurs in a pervaporation device comprising at least one separation membrane wherein a vacuum is applied and/or a purge gas is introduced, thereby allowing the one or more organic solvents to diffuse through said membrane. Consequently, the organic solvents or at least part of the organic solvents are removed and separated from the remaining carrier formulation.

In an embodiment, said purge gas is different from the solvent. In preferred embodiments said gas is nitrogen.

In an embodiment, at least part of the aqueous solution is removed from the formulation during the pervaporation step.

In an embodiment, said organic solvent or part of said organic solvent is allowed to evaporate from the surface of said membrane.

In embodiments, a part of the aqueous solvent that comprises the carriers is allowed to evaporate from the surface of said membrane, for example along with the organic solvent as a natural occurrence to the pervaporation process or intentionally, to change the concentration of the nanoparticles in the aqueous solvent/solution.

In an embodiment, said membrane is a silicone or polydimethylsiloxane (PDMS) membrane. In an embodiment, said membrane is a porous membrane.

In an embodiment, said membrane is sandwiched between one or more metal sheets or plates. In an embodiment, said pervaporation device comprises a plurality of membrane stacks, said membrane stacks comprise a membrane sandwiched between one or more metal sheets of plates.

In an embodiment, a vacuum is applied during said pervaporation process, wherein said pressure is between 0.5 mbar to 100 bar. For example, between 0.5 mbar to 100 mbar, between 1 mbar to 50 mbar, between 5 mbar to 10 mbar and all the ranges and subranges therein between.

In an embodiment, said pervaporation process occurs at a temperature ranging between 20° C. and 35° C. For example, the pervaporation process occurs at a temperature between 21° C. and 35° C., 22° C. and 35° C., 23° C. and 35° C., 24° C. and 35° C., 25° C. and 35° C., and all the ranges and sub-ranges therebetween. In preferred embodiments, the temperature for the pervaporation process is 25° C.

In an embodiment, the flow rate of the feed during pervaporation is between 10 uL and 10 L/min. For example, the flow rate can be between 10 μL/min and 10 L/min, 10 μL/min and 9 L/min, 10 μL/min and 8 L/min, 10 μL/min and 7 L/min, 10 μL/min and 6 L/min, 10 μL/min and 5 L/min, 10 μL/min and 4 L/min, 10 μL/min and 3 L/min, 10 μL/min and 2 L/min, 10 μL/min and 1 L/min, 10 μL/min and 0.5 L/min and all ranges and subranges therein between.

In a preferred embodiment, the flow rate of the feed during pervaporation is between 10 uL and 500 ml/min. For example, the flow rate can be between 10 μL/min and 500 ml/min, 10 μL/min and 400 mL/min, 10 μL/min and 300 mL/min, 10 μL/min and 200 ml/min, 10 μL/min and 100 ml/min, 10 μL/min and 50 mL/min, 10 μL/min and 5 mL/min and all ranges and subranges therein between.

In embodiments, said one or more carriers have a size of at most 10 microns, more preferably at most 1 micron. In embodiments, the carriers preferably have a size of 10 microns to 1 nano micron, 8 micron to 1 nano micron, 6 micron to 1 nano micron, 5 micron to 1 nano micron, 4 micron to 1 nano micron, 3 micron to 1 nano micron, 2 micron to 1 nano micron, more preferably from 1.5 micron to 1 nano micron.

In an embodiment, the carriers as described herein are lipid-based carriers, polymeric carriers or hybrid carriers. Said carriers can be nanocarriers having a size of between 1 nano micron to 1000 nano micron, preferably having a size of 1 nano micron to 900 nano micron, 1 nano micron to 800 nano micron, 1 nano micron to 700 nano micron, 1 nano micron to 600 nano micron, 1 nano micron to 500 nano micron, 1 nano micron to 400 nano micron, 1 nano micron to 300 nano micron, 1 nano micron to 200 nano micron, 1 nano micron to 100 nano micron, 1 nano micron to 50 nano micron, 1 nano micron to 10 nano micron or having a size of 10 nano micron to 1000 nano micron, 50 nano micron to 1000 nano micron, 100 nano micron to 1000 nano micron, 200 nano micron to 1000 nano micron, 300 nano micron to 1000 nano micron, 400 nano micron to 1000 nano micron, 500 nano micron to 1000 nano micron, 600 nano micron to 1000 nano micron, 700 nano micron to 1000 nano micron, 800 nano micron to 1000 nano micron, 900 nano micron to 1000 nano micron or any range therein between.

In further embodiments, carriers are liposomes, lipid nanoparticles, nanostructured lipid carriers, nanoemulsions, polymer nanoparticles, polymer micelle or dendrimers. In a preferred embodiment, said carriers are lipid nanoparticles.

In an embodiment, said carriers are lipid-based carriers. Said lipid-based carriers comprise one or more lipids. The one or more lipids can be in solid and/or liquid form. Said lipid-based carriers may be LNPs, lipoplexes, liposomes, phospholipid micelles, solid lipid nanoparticles, nanostructured lipid carriers or nano-emulsions. Lipid-based carriers useful according to the invention include, for example, cationic lipids, liposomes, in particular cationic liposomes, and micelles, and nanoparticles. Cationic lipids may form complexes with negatively charged nucleic acids. Any cationic lipid may be used according to the invention. Liposomes are phospholipid and cholesterol self-assembled bilayer membranes that enclose an aqueous core, where hydrophilic molecules can be incorporated. Hydrophobic compounds can also be incorporated in the lipid bilayer. Liposomes can be classified in (i) small unilamellar vesicles (SUVs); (ii) large unilamellar vesicles (LUVs) and (iii) multilamellar vesicles (MLVs), according to their size and lamellarity. Solid lipid nanoparticles (SLNs) have a spherical shape with an average diameter of 10-1000 nm. They are used as a colloidal NP drug delivery system in which lipid drug carrier solidifies at room temperature as well as at body temperature. Different solid lipids can be exploited to produce SLNs, such as, tripalmitin, cetyl alcohol, cetyl palmitate, glyceryl monostearate, trimyristin, tristearin, stearic acid, etc. SLNs comprise of solid lipid, such as triglycerides, fatty acids, waxes, partial glycerides, and polyethylene glycosylated lipid; emulsifiers, such as polysorbates, poloxamer and lecithin; and water. Nanostructured lipid carriers (NLC), comprise a blend of solid and liquid lipids which results in a partially crystallized lipid system and many have advantages such as enhanced drug loading capacity, drug release modulation flexibility and improved stability.

In an embodiment, said lipids are chosen from one or more cationic and/or ionizable lipids, one or more helper lipids, one or more sterols, one or more PEGylated lipids or any combination of any of the foregoing.

In an embodiment, said lipid-based carrier is a lipid nanoparticle. Solid lipid nanoparticles (SLNs, sLNPs), or lipid nanoparticles (LNPs), are nanoparticles composed of lipids that are suited to be used as a drug delivery vehicle for drug compounds, especially polynucleotides such as RNA or DNA.

In an embodiment, said LNPs comprise one or more cationic and/or ionizable lipids, one or more helper lipids, one or more sterols, one or more PEGylated lipids or any combination of any of the foregoing. In a preferred embodiment, the LNPs comprise one or more cationic and/or ionizable lipids, one or more helper lipids, one or more sterols, and one or more PEGylated lipids.

In the context of the present disclosure any type of ionizable lipid can suitably be used.

The LNPs may comprise any permanently cationic or cationisable lipid (i.e. any of a number of lipid species which carry a net positive charge at a selective pH, such as physiological pH) or any combination thereof.

In an embodiment, said permanently cationic or cationisable lipids include cleavable lipids.

In an embodiment, said permanently cationic or cationisable lipids are sterol-based cationic lipids, such as DC-cholesterol (3β-[N—(N′,N′-dimethylaminoethane)-carbamoyl]cholesterol) or ICE (imidazole cholesterol ester).

In an embodiment, said LNPs comprise both a cationisable and a permanently cationic lipid. In an embodiment, said LNPs comprise DODAP (cationisable) and DOTAP (permanently cationic) as cationic lipids.

Besides one or more cationic or ionizable lipids, the LNPs further comprise one or more helper lipids. Helper lipids modulate the structure of the LNPs.

In an embodiment, said helper lipids are selected from the group consisting of phosphatidylcholines (PCs), phosphatidylethanolamines (PEs) and sphingolipids (such as glycosphingolipids or sphingomyelins). The term “glycosphingolipids,” as used in this context, pertains to both glycosphingolipids (which bear an O-linked saccharide group) as well as analogs that bear an O-linked polyhydric alcohol group. The term “sphingomyelins,” as used in this context, pertains to both sphingomyelins (which bear a phosphocholine group) as well as sphingomyelin analogs which bear an analog of a phosphocholine group. These compounds may be conveniently described as “sphingomyelins and other sphingoid-based phospholipids”.

The use and inclusion of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the lipid nanoparticle formulations as disclosed herein.

In another embodiment, said carriers are polymeric nanocarriers, such as polymeric nanoparticles, polymeric micelles, dendrimers or hydrogel nanoparticles.

Polymeric nanoparticles are submicron spherical entities composed by a polymeric compact net than can either constitute a polymeric matrix—in the case of nanospheres—or a polymeric wall surrounding a vesicular core—nanocapsules. Polymeric nanoparticles can transport hydrophilic and hydrophobic molecules either entrapped in the polymeric matrix or core or adsorbed to their surface wherein said polymeric nanoparticles may be nanospheres or nanocapsules. Polymeric nanospheres consist in a polymeric matrix in which the drug or cargo is homogenously dispersed, whereas nanocapsules are vesicular systems formed by a polymer wall that surrounds a core containing the cargo. Polymeric micelles are self-assembled spherical nanocarriers formed by amphiphilic block copolymers. In aqueous medium, the block copolymers arrange themselves in a disposition where the most hydrophobic parts of their chains form a hydrophobic core—where hydrophobic molecules can be incorporated—, and the most hydrophilic regions of the polymer chain are displayed outoward. Dendrimers are hyperbranched nanocarriers formed by a central core, branching monomers and functionalized peripheral groups. Dendrimer synthesis can start from the core element (divergent polymerization) or from the peripheral branching units (convergent polymerization), resulting in a structure with a hydrophilic surface and a hydrophobic central core. Molecules can be transported by dendrimers either incorporated in the core and branches, either conjugated to the terminal groups.

In an embodiment, said carriers are metal and inorganic nanocarriers. Said metal and inorganic nanocarriers may be gold nanoparticles, nanoshells, magnetic nanoparticles and quantum dots.

In embodiments, said carriers are comprising, encapsulating, bound to, or adsorbing the one or more active pharmaceutical ingredients (API). As mentioned, the carriers can be used as a delivery system of these APIs.

In an embodiment, said active pharmaceutical ingredient is selected from the group consisting of a polynucleotide, a chemical compound, a polypeptide, a small molecule and any combination thereof. It will be clear to a skilled person that the term “polypeptide” encompasses any type of peptide, including a protein or an antibody, In a preferred embodiment, said active pharmaceutical ingredient comprises a polynucleotide. A “polynucleotide” as defined herein is a combination of nucleotide monomers which are connected to each other through covalent bonds. A single polynucleotide molecule consists of 14 or more monomers of nucleotide in a chain structure. DNA (deoxyribonucleic acid) and RNA (ribonucleic acid) are examples of polynucleotides. In some embodiments, the one or more polynucleotides, are DNA molecules. In some embodiments, the one or more polynucleotides, are RNA molecules.

In embodiments, polynucleotide as active pharmaceutical ingredient can be a double stranded, a single stranded, natural or a synthetic polynucleotide.

In a particularly preferred embodiment, said active pharmaceutical ingredient is one or more RNA molecules.

In some embodiments, the RNA molecules may include long-chain RNA, coding RNA, non-coding RNA, long non-coding RNA, single stranded RNA (ssRNA), double stranded RNA (dsRNA), linear RNA (linRNA), circular RNA (circRNA), messenger RNA (mRNA), self-amplifying mRNA (SAM), Transamplifying mRNA, RNA oligonucleotides, antisense oligonucleotides, small interfering RNA (siRNA), small hairpin RNA (shRNA), antisense RNA (asRNA), CRISPR/Cas9 guide RNAs, riboswitches, immunostimulating RNA (isRNA), ribozymes, aptamers, ribosomal RNA (rRNA), transfer RNA (tRNA), viral RNA (vRNA), retroviral RNA or replicon RNA, small nuclear RNA (snRNA), small nucleolar RNA (snoRNA), microRNA (miRNA), transcription start site-associated (TSSa-)RNAs, upstream antisense (ua) RNAS, promoter upstream transcripts (PROMPTs), or a combination thereof. In some embodiments, the RNA molecules comprise at least one chemical modification comprising backbone modification, sugar modification, or base modification. In this context, a modified RNA molecule comprises nucleotide modifications, e.g. backbone modifications, sugar modifications or base modifications. A sugar modification in connection with the present disclosure is a chemical modification of the sugar of the nucleotides of the RNA molecule. Furthermore, a base modification in connection with the present disclosure is a chemical modification of the base moiety of the nucleotides of the RNA molecule. In this context, nucleotide modifications are selected from nucleotide modifications that are applicable for transcription and/or translation. In further embodiments, the modified RNA comprises nucleoside modifications selected from 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, pyrrolo-cytidine, inosine, α-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, α-thio-adenosine, 8-azido-adenosine, 7-deaza-adenosine, and a combination thereof.

According to the present disclosure, the term “RNA” includes and preferably relates to “mRNA” which means “messenger RNA” and relates to a “transcript” which may be produced using DNA as template and encodes a peptide or protein.

In a preferred embodiment, said polynucleotide encodes an antigen, preferably an antigen linked to an infectious disease or agent.

Higher temperatures are expected to increase the efficiency of the pervaporation process. In embodiments, said formulation comprising one or more carriers is heated before and/or during the pervaporation process. In embodiments, the formulation heated to a temperature ranging between 20° C. and 60° C. For example, to a temperature between 21° C. and 50° C., 22° C. and 45° C., 23° C. and 40° C., 24° C. and 38° C., 25° C. and 35° C., and all the ranges and sub-ranges therebetween. In preferred embodiments, the formulation's temperature is 25° C.

In an embodiment, and after removal of said one or more organic solvents, said formulation comprising carriers are collected and optionally diluted.

In an embodiment, the carrier formulation after solvent removal a pH of between 3 and 8.5, preferably between 6 and 8.5, between 7.2 and 8, more preferably between 7.2 and 7.8 such as 7.4. If required, the pH after pervaporation can be adjusted by means known in the art. In some embodiments, pH can be adjusted before pervaporation.

In an embodiment, said pervaporation process is a continuous process. In an embodiment, said pervaporation process generates a continuous process of manufacturing said formulation. Consequently, a continuous feed of carriers comprising said one or more organic solvents to be removed is provided to the pervaporation device for processing.

In a second aspect, said invention is also directed to a system for manufacturing a carriers, wherein said system comprises one or more mixing units fluidly connected to a pervaporation device.

In embodiments, the system for manufacturing a formulation comprising one or more carriers, wherein said carriers are encapsulating, bound to, or adsorbing one or more active pharmaceutical ingredients, wherein, said system comprises one or more mixing units suited to mix at least one solution comprising one or more active pharmaceutical ingredients with a composition of one or more organic solvents comprising one or more lipids and/or one or more polymers thereby forming a formulation comprising one or more carriers, wherein said one or more mixing units are fluidly connected to a pervaporation device for removal of at least a part of the organic solvents from said formulation.

In embodiments, said system is suited for both laminar and chaotic mixing.

In embodiments, said one or more mixing units used in said system are microfluidic mixing units.

In embodiments, said microfluidic mixing unit comprises an assembly of a plurality of microfluidic mixers in parallel. In embodiments, a microfluidic mixing unit comprises 5 to 20 of said mixers such as 5 to 20, 6 to 19, 7 to 18, 8 to 17, 9 to 16, 10 to 15, 11 to 14 or 12 to 13 microfluidic mixers, preferably 10 microfluidic mixers.

In embodiments, said microfluidic mixing unit comprises a microfluidic chip comprising an assembly of a plurality of microfluidic mixers in parallel, preferably 5 to 20 of said mixers. For example, said microfluidic chip comprises 5 to 20, 6 to 19, 7 to 18, 8 to 17, 9 to 16, 10 to 15, 11 to 14 or 12 to 13 microfluidic mixers, preferably 10 microfluidic mixers.

In embodiments, said a pervaporation device of said system is comprising at least one separation membrane for the removal of one or more organic solvents from a formulation comprising one or more carriers.

In an embodiment, said pervaporation device is in fluid connection to the outlet of one or more mixing units, suited to mix an aqueous solution comprising one or more polynucleotides of interest with a composition comprising one or more lipids and one or more organic solvents, thereby forming a formulation comprising one or more lipid-based carriers.

In an embodiment, said pervaporation device comprises at least one pervaporation unit comprising at least one separation membrane.

In an embodiment, said pervaporation device comprises at least one separation membrane. In a further embodiment, wherein said separation membrane is sandwiched between metal plates or in a single-use plastic holder.

In embodiments, the pervaporation device comprises a plurality of stacks of pervaporation units wherein said each unit comprises a separation membrane sandwiched between one or more metal plates.

In an embodiment, said pervaporation device comprises a plurality of stacks of a separation membrane sandwiched between one or more metal plates.

In an embodiment, said pervaporation device is connected to one or more vacuum pumps, preferably to vacuum pumps with a condenser.

In further embodiments, said pervaporation device comprises and/or connected to one or more condensers. In embodiments, the condensers are vapor condensers.

In embodiments, said pervaporation device is in fluid connection to the inlet of one or more filters for further purification of the solution comprising carriers, wherein said filters preferably have pore sizes ranging from 0.1 μm to 2 μm, more preferably pore sizes from 0.1 μm to 1.5 μm, 0.15 μm to 1 μm, 0.2 μm to 0.8 μm and all ranges and subranges therein between.

In embodiments, said system is thermoregulated to maintain the temperature constant and counterbalance the endothermic evaporation process.

In specific embodiments, said system comprises heating and control means for avoiding temperature decrease, which is caused by the endothermic evaporation of the solvent. In embodiments, said heating is applied to maintain a temperature between 20° C. and 35° C., 22° C. and 35° C., 23° C. and 35° C., 24° C. and 35° C., 25° C. and 35° C., and all the ranges and sub-ranges therebetween.

In embodiments, said system comprises a plurality of T junctions wherein two or more tubing are joined and/or split. Said T junctions are configured to receive reagents, excipients and/or buffers required for manufacturing of one or more carriers.

In embodiment, T junctions may further comprise chambers for an effective addition of reagents, excipients and/or buffers to the system.

In embodiments, said system comprises a plurality of chambers wherein each chamber is configured to receive reagents, excipients and/or buffers required for manufacturing of one or more carriers. Said chamber may or may not be be located at a T junction.

In embodiments, said chambers and/or T junctions are used to adjust the pH of the solution containing carriers.

In an embodiment, said chambers and/or T junctions are located before the mixing unit, between the mixing unit and pervaporation device, after the pervaporation device, between the pervaporation device and one or more filters and/or after the filters.

In embodiments, said system comprises a storage unit for storing one or more reagents, said storage unit can be cooled to a temperature below 10° C., preferably to 4° C.

In embodiments, said system comprises one or more SP-TFF (Single-Pass Tangential Flow Filtration) units. Said one or more SP-TFF units can be placed after microfluidic mixing unit and/or after pervaporation unit.

In an embodiment, said storage unit is in fluid connection with a pump system, such as a peristaltic pump or a syringe pump.

In an embodiment, said system is provided in a cabinet, preferably a wheeled cabinet. In specific embodiments, said cabined is a laminar flow hood.

In an embodiment, said system can be connected to a system for the in vitro transcription of RNA.

The present invention will be now described in more details, referring to examples and figures that are not limitative.

### EXAMPLES

The present invention will now be further exemplified with reference to the following examples. The present invention is in no way limited to the given examples or to the embodiments presented in the figures.

Preparation of LNPs with Pervaporation of Fluid Containing LNPs

LNPs were prepared by microfluidic mixing of an ethanol solution containing an ionizable lipid, DSPC, cholesterol and DMG-PEG2000 at a molar ratio of 50:10:38.5:1.5 with an acetate buffer at pH 5 containing RNA at a concentration of 133 ng/L. The two phases were mixed at a flow rate ratio of 3:1 (water:ethanol). After fabrication, these LNPs were pervaporated by flowing the suspension in a pervaporation unit and applying a vacuum of 10 mBar. The total flow rate was varied between 48 and 100 μL/min and the temperature was varied between 25° C. and 35° C.

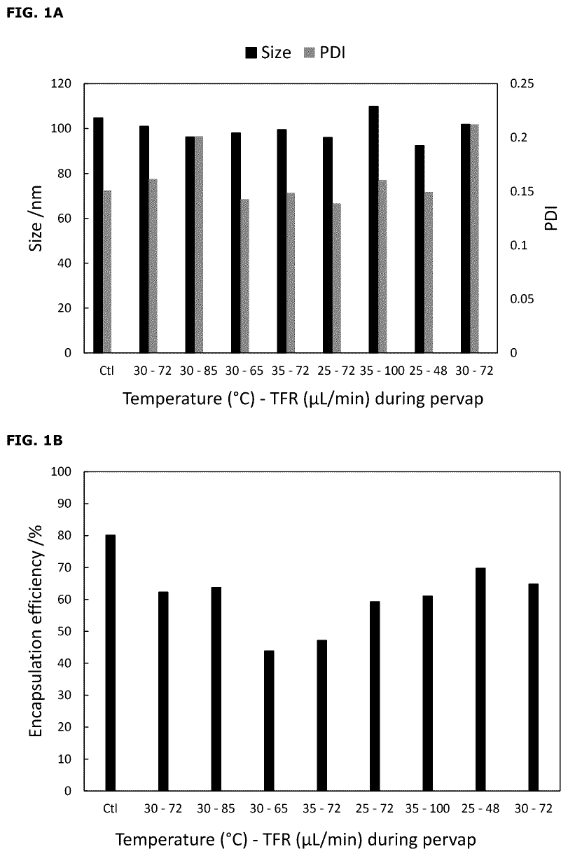

After pervaporation, LNPs were collected and dialyzed to reach a pH of 7.4. The size, PDI and encapsulation efficiency were measured and compared to LNPs not pervaporated using a standard laboratory procedure. Results (see FIG. 1A) show that size is maintained around 100 nm diameter and the PDI remains below 0.2 for all conditions except at high temperature. FIG. 1B shows RNA encapsulation decreases at high temperatures. Taken together, these results demonstrate that pervaporation at 25° C. maintains the size, PDI and encapsulation efficiency of the LNPs, hence this unit operation is not detrimental to the physicochemical characteristics of LNPs.

Ethanol Content of LNPs after Removal of Ethanol

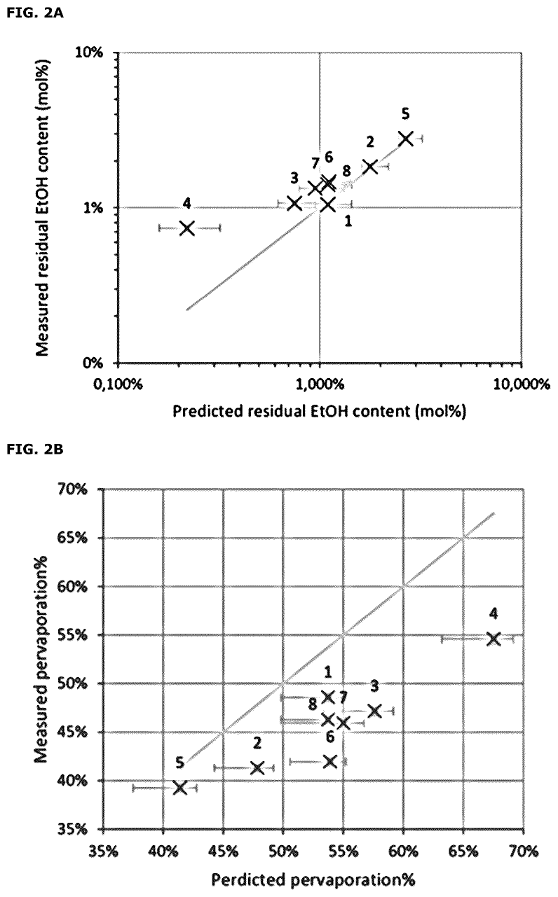

The ethanol content of LNPs and the concentration factor were measured by NMR and by mass balance (weight measurement before and after pervaporation) respectively. Data were plotted against the predicted pervaporation for a system that does not contain any LNP. Results are shown in FIGS. 2A-2B. It appears that the measured ethanol concentration is along the pale bisector line, while the concentration factor is below the pale bisector line. These results indicate that the presence of LNPs causes a selectivity towards ethanol. Hence, lower concentration factor is obtained in the presence of LNPs. They also show that ethanol concentrations ranging from 0.2 to 3% can be obtained. Taken together, these results suggest that a preferred configuration of the whole system is to combine the pervaporation with a single pass TFF (SP-TFF), where the pervaporation removes ethanol and SP-TFF concentrates the solution and removes some of the ethanol left while adjusting the pH.

Functional Evaluation of LNPs with Luciferase Assays

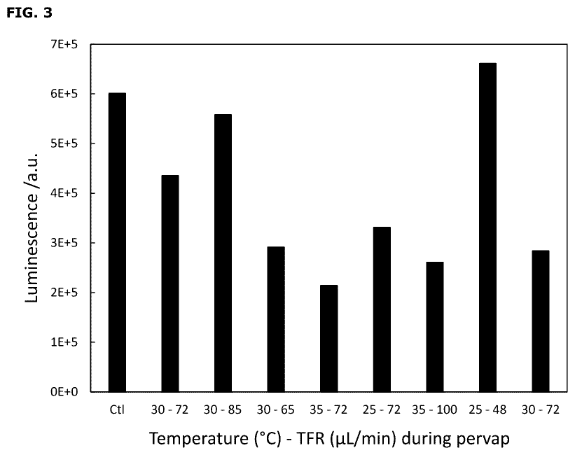

HEK293 cell transfected with LNPs encapsulating RNA encoding for Firefly Luciferase obtained by pervaporation at different total flow rates (TFR) and different temperatures as well as with controls (Ctl) LNPs encapsulating RNA encoding for Firefly Luciferase obtained by using a standard laboratory procedure. Obtained LNPs were subjected to dialysis to adjust the pH before transfection. HEK293 cells transfected with 100 ng of RNA per well in a 96-well plate. Luminescence was measured 24 hours after transfection and results are shown in FIG. 3. Results show that several pervaporation conditions lead to LNPs with similar luminescence, i.e., potency, as LNPs that were not pervaporated and dialyzed using a standard laboratory procedure. This indicates that the bioactivity of LNPs is preserved during the pervaporation step when applying the appropriate conditions of temperature and flow rate.

### FIGURE DESCRIPTION

FIG. 1A shows size and PDI of LNPs that were pervaporated at different total flow rates (TFR) and different temperatures FIG. 1B Encapsulation efficiency of LNPs that were pervaporated at different total flow rates (TFR) and different temperatures. Ctl shows LNPs dialyzed using a standard laboratory procedure.

FIG. 2A Measured vs. predicted residual EtOH content for LNP system pervaporation. The bisector is a gray perpendicular line. X-axis error: ±0.5° C., ±2 mbar, ±2% flowrate; Y-axis error: 5% NMR value FIG. 2B shows measured vs. predicted pervaporation fraction for binary system pervaporation. The bisector is a perpendicular gray line. X-axis error: ±0.5° C., ±2 mbar, ±2% flowrate; Y-axis error: 1% mass value.

FIG. 3 LNPs encapsulating RNA encoding for Firefly Luciferase were pervaporated at different total flow rates (TFR) and different temperatures. LNPs were then dialyzed overnight to adjust the pH. LNPs were then used to transfect HEK293 with 100 ng of RNA per well of a 96-well plate. Luminescence was measured 24 hours after transfection. Ctl shows LNPs not pervaporated and dialyzed using a standard laboratory procedure.

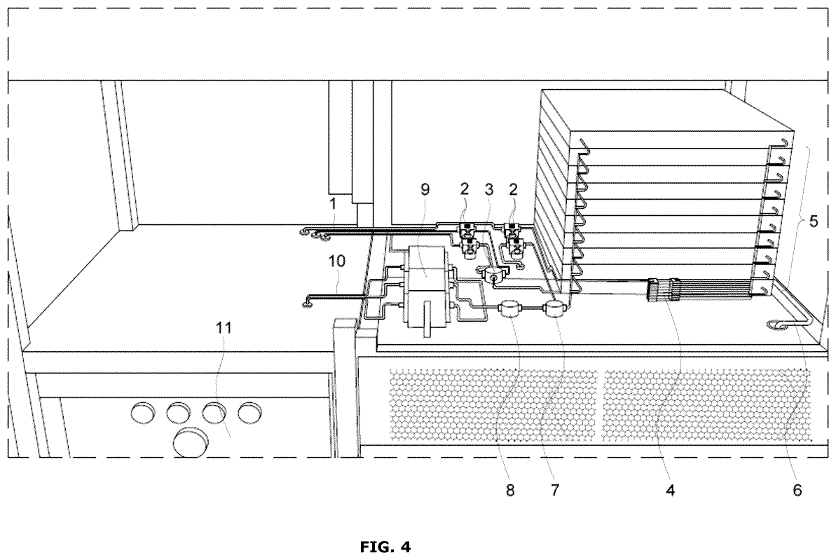

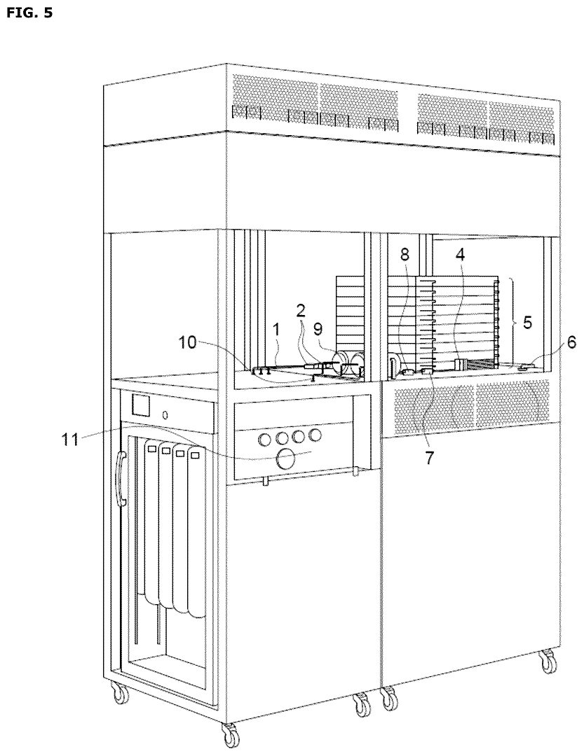

FIG. 4 and FIG. 5 show respectively a front view and a detailed view of an embodiment of a formulation production system for producing carriers according to the current disclosure. The system shown comprises a microfluidic chip (4), stacks of pervaporation units (5), vacuum (6), filters (9), and handling apparatuses: pumps (2), input (reagent) tubing (1), output (product) tubing (10) and chambers (3, 7, 8). Storage containers for reagents and output product for downstream processes can be kept in a cooling department (11), wherein said reagents and output and output is fed to said containers in said cooling department via input (1) and output (10) tubing.

The formulation system shown enables the formulation of biological compounds such as nucleotides in a carrier. To that purpose a cabinet is provided comprising a microfluidic mixing unit (4) is present, which is in fluid connection to a pervaporation unit (5).

In further detail, the system shown comprises a storage unit, preferably a cooling department (11) that can be cooled up to 4° C. The cooling department houses the containers for components such as reagents, buffers, and samples to use in a formulation as well as a unit for storing the compound of interest after processing. The cooling department is in fluid connection with the rest of the system via multiple input tubing (1) which enables the feeding of the system with a required reagent, sample, and/or other compounds for the formulation of a biological compound. The input tubing (1) is connected to one or more pumps (2) to enable the fluid flow in one direction. The far end of the input tubing is connected to a first chamber (3) in which the desired amount and/or number of reagents and samples for the preparation of a biological compound are combined/premixed. For example, the premix can contain nucleic acid, lipid, and encapsulating buffer to form nanoparticles encapsulating nucleic acid.

The first chamber is in fluid connection with a microfluid mixing unit (4) which is then in fluid connection with a pervaporation unit (5). The mixing unit may comprise an assembly of a number of microfluidic mixers in parallel, such as 5 to 20 microfluidic mixers. The microfluidic mixing unit can comprise one or more staggered herringbone micromixers or one or more laminar flow mixers or one or more fractional flow mixers, one or more chaotic mixers. Once the formulation of biological compounds, such as nanoparticles, has formed in the microfluidic mixing unit (4), the fluid comprising the particles is transferred to a pervaporation unit (5) via tubing.

The pervaporation unit (5) comprises one or more stacked pervaporation cassettes in parallel for upscaling the solvent removal from said fluid, for example, 5 to 20 cassettes. Each pervaporation cassette comprises at least one separation membrane. In the shown system, the pervaporation unit is in connection with a vacuum pump (6) to assist the solvent separation via said membrane wherein the separation is done by means of diffusion. Heating is optionally applied before and/or during the pervaporation process to improve the efficiency of solvent separation.

Optionally, one or more SP-TFF units can be added between the microfluidic mixing unit and pervaporation unit to add TFF to the fluid comprising formed particles before its' transfer to the pervaporation unit. The output end of the pervaporation unit is connected to one or more final T-junctions and/or chambers (7 and 8) in serial (one after other) or parallel order. The final T-junctions and/or chambers can be used to adjust the pH of the solution. For example, in the case of producing solution comprising lipid-based carriers pH is adjusted between 3 and 8.5 in the chambers, more specifically to pH 7.4. the chambers can also be used to add excipients to the solution.

The final T junctions and/or chambers (7 and 8) are in fluid connection with the filtering unit (9) where further purification of the solution containing biological composition is performed. To enable such purification the filters can be chosen from but not limited to Acropak (Pall) or Sartopore (Sartorius) filters. The output tubing (10) is connected to the output end of the filtering unit to enable the transfer of the solution with formulated biological compound to one or more containers for storage and/or for the downstream process. Preferably the storage containers are placed in the cooling department (11).

Preferably the cabinet is divided into more than one compartment wherein the different units of the said system are placed. The units of the system are connected to each other to obtain a continuous fabrication process of the product, such as LNPs or other gene delivery vehicles. Preferably the cabinet is on wheels.

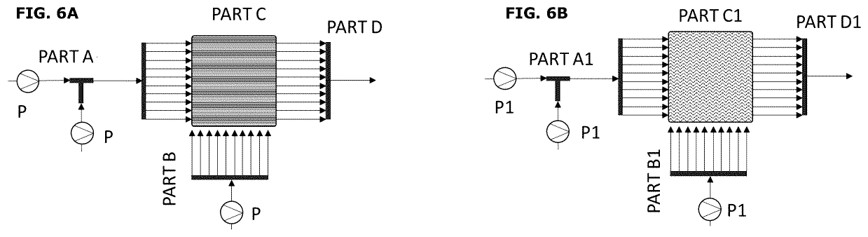

FIG. 6A shows a schematic overview of laminar microfluidic mixing unit

The mixing unit presented induces lamination of the flow-stream for the production of carriers. Unit includes Part A for receiving polynucleotides and a first aqueous solution herein together called as stream 1, Part B for receiving a second stream comprising a second aqueous solvent or an organic solvent. Stream 1 and 2 are introduced into Part C flowing under laminal flow conditions where rapid dilution occurs, and then to Part D where the final product, carriers containing polynucleotides/therapeutic agent, exit the unit. The unit comprises several pumps, represented with P to assist the fluid flow.

FIG. 6B shows a schematic overview of a chaotic microfluidic mixing unit. Unit includes Part A1 for receiving therapeutic agent such as polynucleotides and a first aqueous solution, together called herein as stream 1, Part B1 for receiving a second stream comprising carrier forming materials in a second aqueous solvent or an organic solvent.

The mixing of two streams coming from Part A1 and Part B1 occurs in the zigzagged pattered central channel (Part C1) where the fluids injected are chaotically mixed. The chaotic mixing results in the assembly of carriers containing therapeutic agent which then flows into the Part D1 to exit the unit. The unit comprises several pumps, represented with P1 to assist the fluid flow.

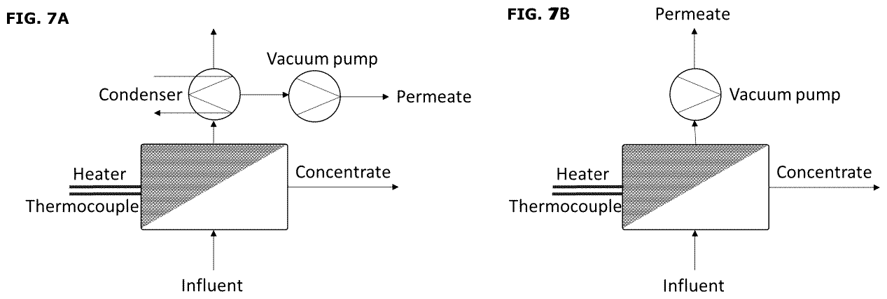

FIG. 7A shows a schematic overview of a pervaporation unit combined with a condenser, whereas FIG. 7B shows a setup without a condenser.

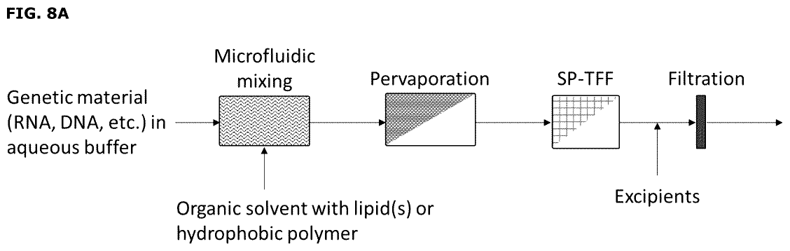

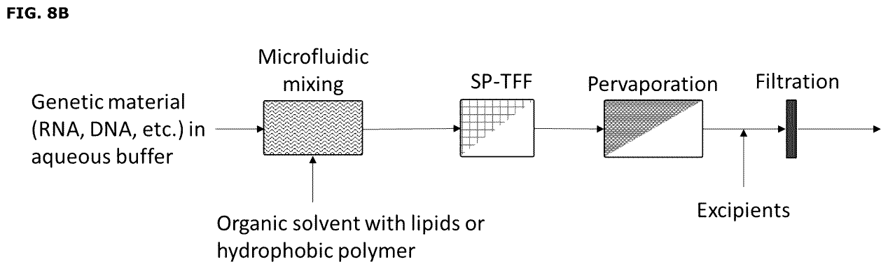

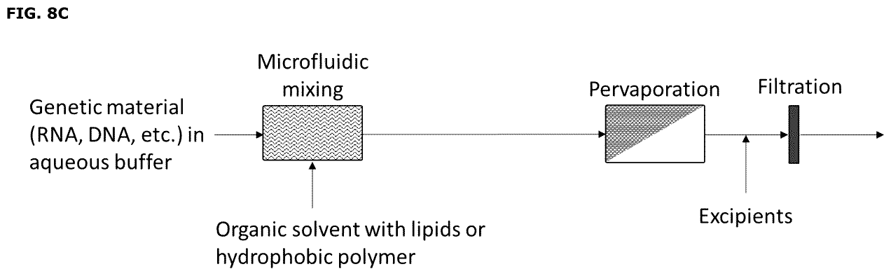

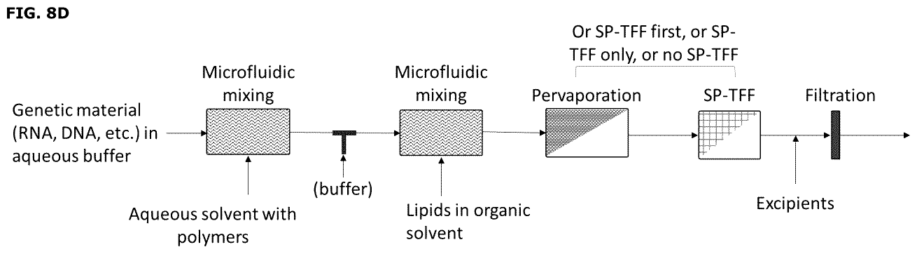

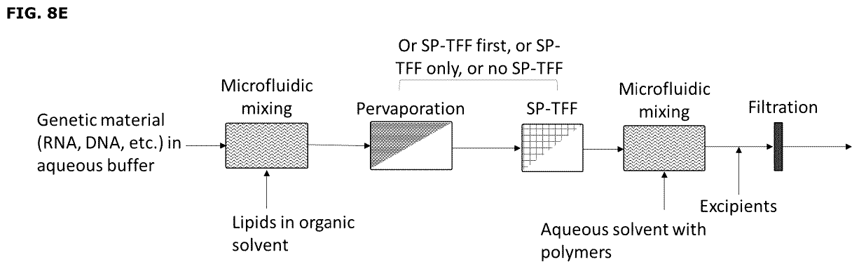

FIG. 8A-FIG. 8E shows different schematic set-ups of possible embodiments of the system of the current invention. In FIG. 8A a set-up comprising microfluidic mixing unit, Pervaporation unit, SP-TFF, excipient inlet, and filtration unit. SP-TFF unit is located between pervaporation unit and filtration unit. In FIG. 8B a set-up comprising microfluidic mixing unit, SP-TFF, Pervaporation unit, excipient inlet, and filtering unit. In FIG. 8C a set-up comprising microfluidic mixing unit, a pervaporation unit, excipient inlet, and filtering unit. In FIG. 8D a set-up comprising a first microfluidic mixing unit, an inlet, a second microfluidic mixing unit, a pervaporation unit, a SP-TFF unit, excipient inlet, and a filtering unit. In FIG. 8E a set-up comprising a first microfluidic mixing unit, a pervaporation unit, a SP-TFF unit, a second microfluidic mixing unit, an excipient inlet, and a filtering unit.

It is clear that the method according to the invention, and its applications, are not limited to the presented examples and/or Figures.

The present invention is in no way limited to the embodiments described in the examples and/or shown in the figures. On the contrary, methods according to the present invention may be realized in many different ways without departing from the scope of the invention.

### FIGURE ELEMENTS

1: input tubing
2: pumps
3: first chamber at a T junction
4: microfluidic chip
5: pervaporation unit
6: vacuum
7: final chamber at a T junction
8: final chamber
9: filters
10: output tubing
11: cooling department

1. A method for manufacturing a formulation of one or more lipid nanoparticle, polymeric, or hybrid carriers comprising one or more active pharmaceutical ingredients, said method comprising:
a) mixing a solution of one or more active pharmaceutical ingredients with one or more organic solvents comprising one or more lipids and/or one or more polymers, thereby creating a mixture, wherein said one or more lipids are selected from the group consisting of one or more cationic and/or ionizable lipids, one or more helper lipids, one or more sterols, one or more PEGylated lipids, or any combination thereof; and subsequently
b) removing at least a portion of said one or more organic solvents from said solution using a pervaporation process.

2. The method of claim 1, wherein said pervaporation process generates a continuous process of manufacturing said formulation.

3. The method of claim 1, further comprising yielding a single-phase liquid mixture comprising said lipid nanoparticle, polymeric, or hybrid carriers resulted from (a).

4. The method of claim 1, wherein said active pharmaceutical ingredient is selected from the group consisting of a polynucleotide, a chemical compound, a polypeptide, and any combination thereof.

5. (canceled)

6. The method of claim 1, further comprising mixing a further solution comprising one or more polymers with said mixture.

7. The method of claim 1, wherein said pervaporation process is a membrane separation process.

8. (canceled)

9. The method of claim 1, wherein said pervaporation process occurs in a pervaporation device comprising at least one pervaporation membrane; said method further comprising applying a vacuum or a purge gas to said pervaporation membrane under a sufficient condition so that said one or more organic solvents diffuse through said pervaporation membrane.

10. (canceled)

11. The method of claim 1, wherein during said pervaporation process said solution is flowed at a flow rate between 10 μL/min and 10 L/min.

12. The method of claim 11, wherein said flow rate is between 10 μL/min and 500 mL/min.

13. The method of claim 1, wherein said one or more organic solvents are selected from the group consisting of ethanol, methanol, propanol, isopropanol, butanol, and mixtures thereof.

14. The method of claim 1, wherein said mixing said solution comprises microfluidic mixing of said solution.

15. The method of claim 1, wherein said carriers have a size of at most 10 microns.

16. (canceled)

17. The method of claim 1, further comprising reducing shear stress on said one or more carriers.

18. The method of claim 1, wherein said pervaporation process does not comprise utilizing a buffer.

19. A system for manufacturing a formulation comprising one or more carriers with one or more active pharmaceutical ingredients, said system comprising:
a) one or more mixing units adapted to mix at least one solution comprising one or more active pharmaceutical ingredients with a composition of one or more organic solvents comprising one or more lipids and/or one or more polymers thereby forming a formulation comprising one or more carriers, and
b) a pervaporation device; wherein said one or more mixing units are fluidly connected to said pervaporation device for removal of at least a part of said organic solvents from said formulation.

20. The system of claim 19, wherein said one or more mixing units comprises one or more microfluidic mixing units.

21. The system of claim 20, wherein each of said one or more microfluidic mixing units comprises an assembly of a plurality of microfluidic mixers in parallel.

22. The system of claim 19, wherein said pervaporation device comprises at least one separation membrane.

23. The system of claim 19, wherein said pervaporation device comprises a plurality of stacks of pervaporation units, wherein each of said pervaporation units comprises a separation membrane sandwiched between one or more metal plates.

24. The system of claim 19, further comprising one or more condensers.
